(19) United States
(12) Patent Application Publication
(10) Pub. No.: US 2024/0345056 A1
(43) Pub. Date: Oct. 17, 2024

(54) SYSTEMS AND METHODS FOR ANALYZING POND WATER HEALTH

(71) Applicants: JORGE E. LOPEZ DE CARDENAS, SUGAR LAND, TX (US); TIMOTHY AL ANDRZEJAK, SUGAR LAND, TX (US); ANTHONY FRANK VENERUSO, SUGAR LAND, TX (US)

(72) Inventors: JORGE E. LOPEZ DE CARDENAS, SUGAR LAND, TX (US); TIMOTHY AL ANDRZEJAK, SUGAR LAND, TX (US); ANTHONY FRANK VENERUSO, SUGAR LAND, TX (US)

(21) Appl. No.: 18/589,765

(22) Filed: Feb. 28, 2024

Related U.S. Application Data
(60) Provisional application No. 63/487,378, filed on Feb. 28, 2023.

(51) Int. Cl. G01N 33/18

(52) U.S. Cl. CPC G01N 33/1806; G01N 33/1886

(57) ABSTRACT

Systems and methods for analyzing pond water health are provided herein. A system for analyzing pond water health includes one or more of an oxidation-reduction potential sensor, a dissolved oxygen sensor, or an H₂S gas sensor. The system further includes a housing containing a control module, wherein the control module is in informational communication with the sensor(s). The system is positionable over a surface of a pond, proximal to the surface, or adapted to float on the surface. A method for analyzing pond water health of water pond includes positioning the system over or on a surface of the pond, obtaining data from the sensor(s) using the control module, and generating an alert using data from the control module based on a determination that pond health is in decline.

### CROSS-REFERENCE TO RELATED APPLICATIONS

This application claims the benefit of U.S. Provisional Application No. 63/487,378, filed Feb. 28, 2023.

### TECHNICAL FIELD

The technical field generally relates to systems and methods for analyzing the health of water stored in open ponds, e.g., oilfield produced water resources, and more particularly relates to systems and methods to analyze conditions for bacteria growth within produced water resources stored in open-air water pits.

### BACKGROUND

Both oil and water are typically produced from a given oil well. The oil and water are separated, leaving substantial quantities of “produced water” that is conventionally disposed or remediated. The produced water is typically referred to as “brine” owing to its typically elevated salinity levels, possible presence of dissolved solids or minerals and often the presence of various organic chemical species. The produced water is a by-product of oilfield operations and is generally not directly usable without remediation. Disposal of the produced water typically involves injecting the water back underground using a disposal well. Alternatively, the produced water, or a portion of it, is treated for reuse within the oilfield for certain oilfield extraction operations, such as: well drilling, stimulation (e.g., hydraulic fracturing), or water injection.

Additionally, for scenarios where produced water is treated and reused for stimulation operations in oilfield reservoirs, excessive anaerobic bacteria content in the produced water can potentially lead to biofouling of an oilwell. Biofouling refers to the inadvertent introduction of biological species into a well whereby subsequent growth of the biological species over time reduces permeability and porosity within the reservoir and impedes the flow of petroleum into the well, thus reducing yield from the well. Anaerobic bacteria are of particular concern for biofouling since, unlike aerobic bacteria, anaerobic bacteria are able to thrive in oxygen-depleted conditions such as those found within hydrocarbon reservoirs. Fluids injected into reservoirs are typically chemically treated in-line prior to exposure into the reservoir to minimize biofouling. Nonetheless, monitoring and control of anaerobic bacteria in produced water intended for reuse remains important to help reduce the likelihood of biofouling.

Owing to the large volumes of produced water regularly produced within the oilfield, the conventional practice requires storage of produced water in open-air water ponds regardless of whether it will be disposed or remediated for reuse. FIGS. 1 and 2 show the typical structure of an open-air water storage pond 1 employed in oilfields. A given water pond may include a berm 3 that encircles a water resource 2. Many times, the water pond may be deeper than ground level 4. Additionally, the bottom of the pond and internal sides of the berm 3 are typically covered with a liner 5, which is an impermeable tarp-like material that helps prevent seepage of water into the ground.

Oilfield water ponds are typically incorporated into a network, whereby water ponds are connected by permanent pipelines or temporary flex pipe to transfer water among the ponds. The networking nature of the ponds allows for access to storage capacity over a larger area of the oilfield. For example, if one region of an oilfield has elevated drilling activity or a higher ratio of produced water-to-oil, then produced water storage ponds in that region may reach their capacity and restrict oil production.

Produced water naturally contains a high content of minerals with salinity levels that often exceed 50,000-ppm. The high mineral content of produced water supports the growth of bacteria. The growth of anaerobic bacteria colonies is of particular concern owing to the fact that anaerobic bacteria metabolize sulfur-containing minerals to generate hydrogen sulfide (H₂S) gas, which is particularly undesirable for many reasons including health and safety.

When anaerobic bacteria levels become high and H₂S gas is detected, produced water ponds are typically treated with copious amounts of chemical oxidizers such as hydrogen peroxide to kill-off the anaerobic bacteria. Thus, oilfield produced water management operators typically follow a reactionary approach regarding the evolution of H₂S gas. In addition to high costs, chemical treatment after detection of H₂S gas is typically only mildly effective as the anaerobic bacteria are already well established in the water pond and any beneficial effects of the chemical treatment are short-lived.

Oxidation-Reduction Potential (ORP) is a measurement that can be used to identify the ability of a given body of water to cleanse itself by providing an indication of the water's ability to oxidize contaminants. The ORP measurement technique determines the transit of electrons during Oxidation-Reduction reactions. Under oxidizing conditions, an ORP probe loses electrons to the water, creating a positive voltage potential from the ORP probe to the water. Under reducing conditions, electrons are donated to the probe from the water, creating a negative voltage potential. ORP is typically reported in units of millivolts. A low ORP in the water is an indication that the water conditions are conducive to the growth of anaerobic bacteria. Similarly, Dissolved Oxygen (DO) is a measurement that helps determine the health of a given body of water and is typically reported in parts-per-million (ppm). A given body of water is considered healthy if its conditions are conducive to oxidation (i.e., positive ORP measurement or elevated DO values) allowing for the natural elimination or control of anaerobic bacteria. In contrast, a given body of water is considered unhealthy or “sour” if its conditions are conducive to reduction (i.e., negative ORP measurement or near zero DO values) allowing for anaerobic bacteria to flourish unimpeded.

The measurement of gas phase H₂S can be accomplished via a variety of techniques, including gold film sensors, sulfur dioxide (SO₂) conversion sensors, and electrochemical cells, among others. Gold film sensors function by absorbing H₂S gas, causing the gold film to undergo an increase in electrical resistance proportional to the mass of H₂S absorbed. Sulfur dioxide conversion sensors function by using a catalytic reaction to convert H₂S to SO₂ followed by the use of a fluorescence chamber to excite the SO₂ molecules using ultraviolet light. A photomultiplier tube is then used to detect the fluorescence and establish an H₂S concentration. Electrochemical cell detectors utilize electrodes that are surrounded by a permeable membrane, which allows air to diffuse into the cell. In the presence of H₂S, an oxidation-reduction reaction occurs that causes a change in current to be measured and converted into an H₂S concentration.

Water depth measurements are particularly useful to monitor the inflow or extraction of water from the pond. Depth measurements can be obtained through a variety of devices including hydrostatic pressure gauges, reflective level meters, or piezo-resistive pressure sensors, among others. Hydrostatic pressure gauges correlate water depth with the pressure measured at the bottom of a given body of water. Depth can be obtained through reflective level meters by using radar or ultrasonic beams emitted from the water surface that reflect off the bottom of a given body of water. Piezo-resistive pressure sensors use a piezoresistive material, often arranged in a diaphragm or membrane structure. When submerged in water, the diaphragm deforms in response to changes in hydrostatic pressure. This deformation results in mechanical stress on the piezoresistive material which induces a change in its electrical resistance proportional to the change in pressure. The water's depth, H, can then be calculated using the water's density, ρ, and the measured hydrostatic pressure, P, as H=P/ρ. The water's density can be determined by using well known state of the art methods and sensors such as hydrometers, pycnometers, or digital hydrometers.

Monitoring oilfield produced water quality is typically time consuming and labor intensive, often requiring personnel to travel to a given water pond (that may be in a remote location), sample the water, and conduct testing activities either at the pond site or at a laboratory to obtain measurements with sufficient precision. Therefore, diagnostic parameters such as ORP and DO are normally obtained as point measurements only every couple of days. Additionally, the repeatability and accuracy of such point measurements is problematic owing to variability in sampling, handling of the samples, and the use of different sensors, among other factors. Furthermore, elevated wind speeds effectively reduce the local concentration of H₂S gas in the air making it more difficult to detect H₂S sources and emissions when measurements are taken in the vicinity of a produced water pond and mixed-use industrial sites.

Accordingly, it is desirable to provide systems and methods that enable superior analysis of water health in open-air ponds, such as oilfield produced water resources. In addition, it is desirable to provide systems and methods that can monitor the effects of treatments applied to these open-air ponds. Furthermore, it is desirable to provide systems and methods for analyzing open-air ponds that enable prediction of future water health in the ponds. Other desirable features and characteristics of the present disclosure will become apparent from the subsequent detailed description and the appended claims, taken in conjunction with the accompanying drawings and this background.

### BRIEF SUMMARY

Systems and methods for analyzing pond water health are provided herein. In an embodiment, a system for analyzing pond water health includes one or more of an oxidation-reduction potential sensor, a dissolved oxygen sensor, or an H₂S gas sensor. The system further includes a housing containing a control module, wherein the control module is in informational communication with the sensor(s). The system is positionable over a surface of a pond, proximal to the surface, or adapted to float on the surface.

In another embodiment, a method for analyzing pond water health of water pond is provided and includes positioning the system over or on a surface of the pond, obtaining data from the sensor(s) using the control module, and generating an alert using data from the control module based on a determination that pond health is in decline.

### BRIEF DESCRIPTION OF THE DRAWINGS

The various embodiments will hereinafter be described in conjunction with the following drawing figures, wherein like numerals denote like elements, and wherein:

FIG. 1 is a schematic diagram illustrating an oilfield water storage pond.

FIG. 2 is a schematic diagram illustrating an oilfield water storage pond.

FIG. 3 is a schematic diagram illustrating an embodiment of a system for monitoring an oilfield produced water pond.

FIG. 4 is a block diagram illustrating an embodiment of the specific electronic modules and their specific functions that implement an autonomous system for monitoring an oilfield produced water pond.

FIG. 5 is a chart showing ORP and depth measurements of a produced water pond.

FIG. 6 is a flowchart illustrating an embodiment of a system for alerting if pond health is in decline.

FIG. 7 is a chart of ORP measurements showing the effects of water treatments being applied to a produced water pond.

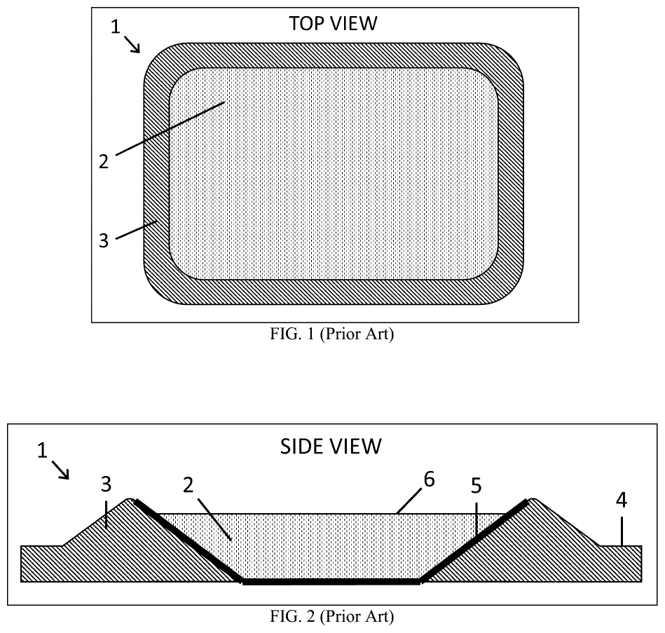

### DETAILED DESCRIPTION

The following detailed description is merely exemplary in nature and is not intended to limit the oilfield water storage systems and methods as described herein. Furthermore, there is no intention to be bound by any theory presented in the preceding background or the following detailed description. The description is not in any way meant to limit the scope of any present or subsequent related claims.

As used here, the terms “above” and “below”; “up” and “down”; “upper” and “lower”; “upwardly” and “downwardly”; and other like terms indicating relative positions above or below a given point or element are used in this description to more clearly describe some embodiments. However, when applied to equipment and methods for use in wells that are deviated or horizontal, such terms may refer to a left to right, right to left, or diagonal relationship as appropriate.

Provided herein are systems and methods for analyzing pond water health. Although the systems and methods described herein mainly apply to open air oilfield produced water ponds, the systems and methods can be applicable to other industries and markets where water is stored in open ponds or tanks and where analysis of the water quality is desired. The systems and methods described herein enable ORP, and/or DO data to be obtained, optionally alongside H₂S gas measurements taken directly over the surface of the water with such measurements possibly gathered multiple times per day. In this regard, an early indication can be generated of when water quality is deteriorating and if a pond is actively generating H₂S gas to enable remediation measures to commence before costs and efforts to remediate the pond escalate. Furthermore, monitoring of some of these parameters, such as ORP, can be used to confirm the effects of treatments applied to a produced-water pond.

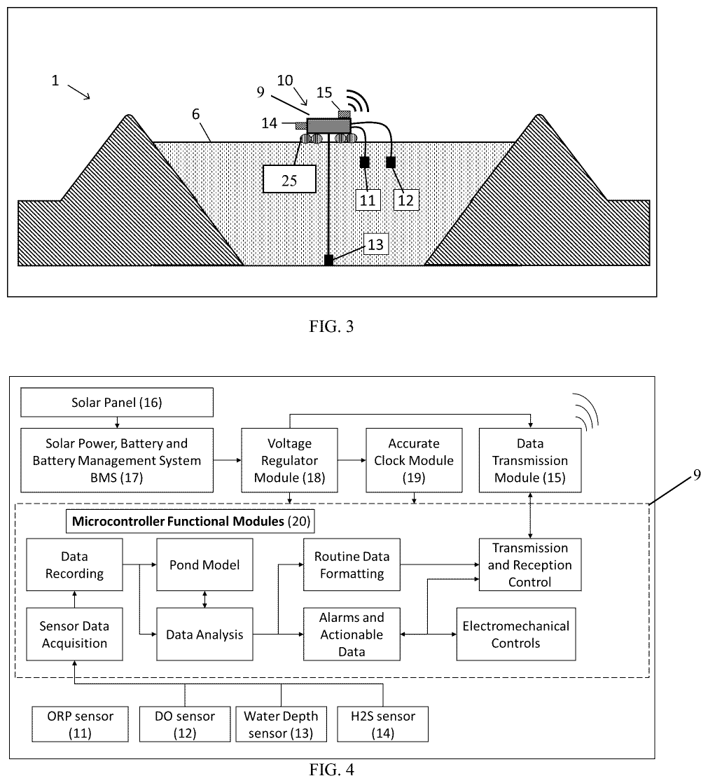

Referring to FIG. 3, an exemplary system 10 for analyzing pond water health is shown. In embodiments, the system 10 may be autonomous, with data gathered in the absence of user prompts. Alternatively, the system 10 may be manually operated with a user controlling gathering of data using the system 10. The system 10 may be positioned over or on a surface 6 of a water pond 1, which may be an open-air pond such as an oilfield water pond. In particular, the system 10 may be suspended over the surface 6 of the oilfield water pond 1, proximal to the surface 6, or may be adapted to float on the surface 6 of the pond 1 using flotation feature(s) 25, e.g., buoys, pontoons, or the like. By “positioned” or “positionable”, it is meant that the system 10 may be retained in the stated position and can be left in position in the absence of human intervention, with the system 10 capable of gathering data while positioned. As depicted in FIG. 3, the system 10 is designed to float on the water surface 6 using buoys 25. Probes 11, 12 and electronics of the autonomous system can be powered with a solar panel and a rechargeable battery. In embodiments, system 10 including the flotation feature(s) allows the system 10 to be easily repositioned and anchored in place to obtain measurements at any position over a given pond 1. This mobility is particularly useful to compare the water quality in different parts of the pond. For example, the water quality in the middle of the pond 1 can be compared with the water quality near inlets where water is being pumped into the pond 1. Additionally, since the data may be obtained based on measurements made directly from the water, in real time with the same sensors used for the respective measurements, physical sampling for remote measurement is not required. Therefore, the system 10 maximizes repeatability and reliability of the measurements.

In embodiments, the system 10 comprises one or more of an oxidation-reduction potential (ORP) sensor 11, a dissolved oxygen (DO) sensor 12, or an H₂S gas sensor 14. The system can include multiple of each of the sensors 11, 12, 14. In embodiments, the system 10 comprises a combination of two or more different types of the above sensors 11, 12, 14. For example, in an embodiment, the system includes at least one ORP sensor 11, and additionally includes at least one DO sensor 12 and/or at least one H₂S gas sensor 14. For example, in an embodiment, the system 10 includes one type of either the ORP sensor 11 and the DO sensor 12, and may include multiple of the respective sensors 11, 12, in addition to at least one H₂S gas sensor 14. The system 10 may further include a water depth sensor 13 in addition to the above-mentioned sensors 11, 12, 14. It is to be appreciated that in embodiments, the system 10 includes both the DO sensor 12 and the ORP sensor 11, although in other embodiments only one type of these sensors 11, 12 are provided. In another embodiment, the system 10 includes at least one ORP sensor 11 and at least one water depth sensor 13.

The ORP sensor 11 and the DO sensor 12 are adapted to submerge within the body of water to provide desired measurements. In this regard, the ORP and DO sensors may be tethered to the housing 9 of the system 10 and allowed to hang beneath the flotation feature(s) when the system 10 is disposed over or on the pond 1. The water depth sensor 13, depending on the type of sensor, may comprise a probe adapted to rest on the floor of the pond or, alternatively, may be positioned adjacent the surface 6 of the pond. The H₂S gas sensor 14 adapted to be positioned proximal to the water surface 6, such as within 3 inches or 12-inches or 24-inches or 36-inches of the water surface 6, when the system 10 is disposed over or on the pond 1.

The system 10 may be capable of recording measurements from the sensors 11, 12, 13, 14, using a control module 20 that is contained within the housing 9 and in informational communication with the sensors 11, 12, 13, 14, at different acquisition rates that can be adjusted as needed, such as every minute or 15 minutes or 30 minutes or every hour or every day. These frequent measurements allow water quality trends to be discernable. Additionally, the system 10 may optionally be equipped with a data transmission module 15 enabling measurements to be observable in real-time from the control module 20, i.e., data may be transmitted immediately or shortly after the data is generated by the control module 20. Data transmission from the system 10 can be accomplished through well-known telecommunications techniques such as radio frequency (RF) signals to cell phone towers or directly to satellites for relay to ground based stations. Although not shown, the data may be transmitted to a remote operator's location, such as a computer, cellphone, or the like, that can be accessed remotely. Alternatively, data may be transmitted from the data transmission module 15 via Bluetooth, radio communication or Wi-Fi with a receiver module (not shown) located near the produced water pond 1. The transmitted data may be populated in a computer program that is run by the remote computer, such as in a database that can be accessed by the remote computer.

Referring to FIG. 4, with continued reference to FIG. 3, an exemplary functional block diagram is shown for the system 10 of FIG. 3, including the sensors 11, 12, 13, 14 as described above as well as an exemplary control module 20 and functions that may be programmed within the control module 20. A solar panel 16 and Battery and Battery Management System (BMS) 17 may provide electrical power to the system 10. A voltage regulator module 18 may be provided to maintain a constant supply voltage to the system 10 throughout variations of the battery's output voltage as it goes through its daily charge/discharge cycles, depending on the availability of solar energy. An accurate clock module 19 may provide a time reference for the system 10 to conduct, record and report its various functions since the system 10 may be autonomous and may not be in direct communication with an outside time reference. An example of such a time reference is “unix time,” which is an internationally recognized date and time representation widely used in telecommunications and computing.

The control module 20 shown in FIG. 4 may perform several functions. In embodiments, the control modules 20 acquires measurements from the sensors 11, 12, 13 and 14 described above and records those measurements in an electronic storage device. Then, as programmed and optionally based on the time reference from the accurate clock module 19, the control module 20 may routinely format and transmit the collected data via the data transmission module 15 to the remote user or database. In embodiments in accordance with an exemplary method, an alert is generated using data from the control module 20 based on a determination that pond health is in decline. In various embodiments, the control module 20 may generate the alerts or, alternatively, the remote computer may generate an alert based on data provided by the control module. For example, in embodiments, the control module 20 autonomously generates and transmits an alert to the remote computer if any of the sensor measurements are outside of preset limits, or expected to exceed preset limits, based on a mathematical model of the pond and the sensor measurements and their history. In embodiments, the control module 20 generates alerts based on rate of change in ORP measurements from the ORP sensor 11, and factors such as pond volume and/or water chemistry may be considered by the control module 20 to generate the alert. Variations and evolution of measurements from the ORP sensor 11 the DO sensor 12, and the H₂S gas sensor over time may depend on the pond's volume and water chemistry. For example, a sudden negative drop in the ORP measurement within a small pond that has had a comfortably high positive ORP value may not be as important as that same drop for a large pond that has a marginal ORP value. In the first case, if the system 10 initiates an alarm to the remote computer, the alarm may be disregarded as unimportant, especially if the ORP quickly returns to a safe level. However, the same sudden negative drop in the ORP within a large pond that has been at a marginal ORP level may be determined to be cause for the system 10 to initiate an alert.

As alluded to above, the system 10 including the flotation feature(s) allows the system 10 to be repositioned and obtain measurements at any position over a given pond. In an embodiment, a method for analyzing pond water health includes comparing data obtained in different parts of the pond. For example, data obtained from in the middle of the pond 1 can be compared with data obtained proximate to inlets where water is being pumped into the pond 1.

In accordance with an exemplary method of analyzing pond water health, ORP and/or DO measurements are taken using the ORP and/or DO sensors, respectively. It is to be appreciated that, in embodiments, the system 10 includes only ORP or DO sensor(s) (i.e., only one type of sensor must be provided in the system 10). ORP and DO measurements can be utilized to determine if conditions within pond 1 are conducive to anaerobic bacteria growth using the control module 20 (with negative ORP values and low DO values corresponding to conditions that are conducive to anaerobic bacteria growth). The ORP and DO measurements, however, do not indicate whether anaerobic bacteria are actively producing H₂S gas. In accordance with embodiments described herein, H₂S gas measurements are obtained from the H₂S gas sensor 14, which is positioned proximal to the water surface 6 as opposed to simply the vicinity of the water pond 1. Obtaining H₂S gas measurements close to the water's surface 6 may provide an early warning that an H₂S problem is arising. In this way, the system 10 provides data that may be employed in accordance with exemplary methods as described here to enable the control module 20 to predict evolution of excessive levels of H₂S and allow action to be taken before unhealthy levels of the H₂S are exceeded in the produced water pond.

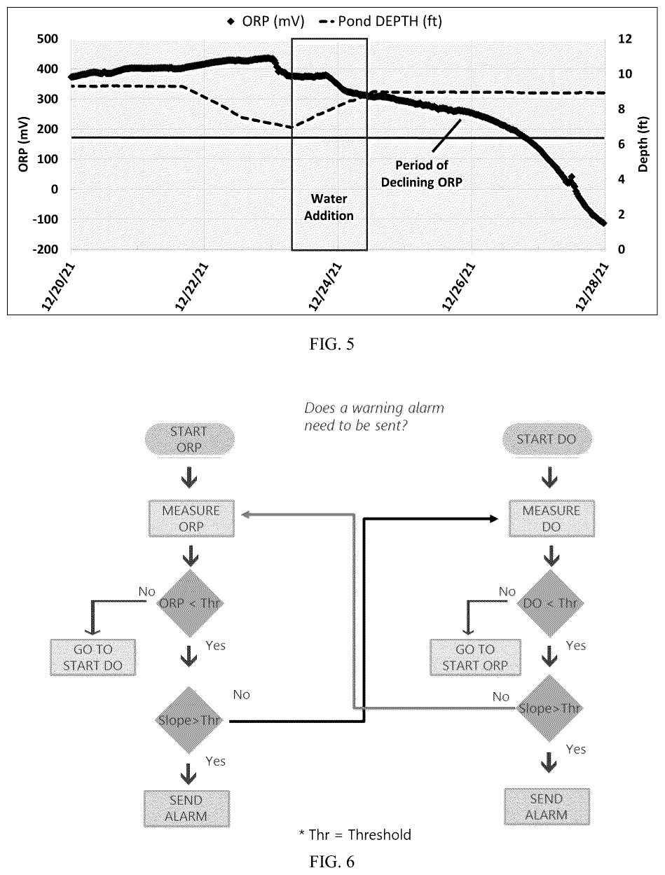

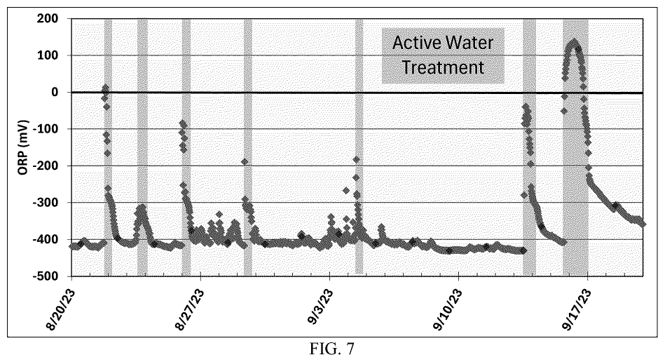

As set forth above, the system 10 may further include the water depth sensor 13 and the method may include considering water depth measurements in combination with data from other sensors 11, 12, 14 to determine water health and, ultimately, generate alerts. Water depth measurements assist in the interpretation of water quality measurement by reflecting activity such as water addition or removal at a produced water pond 1 and, in accordance with an exemplary method, can be employed to render an assessment of water health. For example, when produced water is added to a pond 1, it may contaminate the pond with anaerobic bacteria that may deteriorate pond water health. FIG. 5 provides actual ORP data obtained from an oilfield produced water pond during a field trial. The graph in FIG. 5 reveals that ORP declined over several days after produced water from another source was added to the pond, indicating that anaerobic bacteria colonies were introduced causing the water quality to decline. Thus, in accordance with an exemplary method, an alert may be generated by registering a change in water depth, e.g., an increase of at least 6 inches, alternatively an increase of at least 1 foot, alternatively an increase of from 6 inches to 1.5 feet, in conjunction with a measured decrease in ORP, e.g., a decrease in ORP of at least 20 mV, alternatively a decrease in ORP of from 20 to 50 mV, alternatively a decrease in ORP of from 50 to 100 mV, alternatively a decrease in ORP of from 100 to 200 mV. The alert may be an alarm generated by the remote computer based on acquired data received from the data transmission module 15. Alternatively, the data transmission module 15 itself may generate the alert. The alert may be an e-mail, a text message, or other data communication that provides an indication that a preset combination of water depth and ORP measurement has been exceeded. Alternatively still, the remote computer may generate the alert based on data received from the data transmission module 15. It is appreciated that DO measurements can be used in place of or in addition to ORP measurements in a similar manner as described above to generate an alert that is predictive of impending poor water health.

Obtaining the ORP and/or DO data, along with H₂S measurements and, optionally, water depth in real-time enables proactive treatment of water in the water pond 1 before conditions deteriorate to an unacceptably excessive degree. Conventional remediation may proceed based on the alert, such as the addition of oxidizing chemicals like hydrogen peroxide, sodium hypochlorite, or chlorine dioxide, to avoid scenarios where H₂S gas concentrations in the vicinity of the pond reach a level of concern. In embodiments, the method further includes automatically commencing remediation of the water in the pond 1 based on the alert.

In embodiments, the ORP and/or DO data, along with H₂S measurements and, optionally, water depth can be further utilized to automate the management of produced water ponds by sending a trigger signal to chemical dosing equipment (not shown) located on-site at the water pond 1. In embodiments, when it is determined that threshold values in the ORP, DO, and/or H₂S gas measurements are surpassed, a signal is generated, either from the system 10 (communicated by the data transmission module) or from the remote computer, to the chemical dosing equipment (not shown). The chemical dosing equipment may then release treatment chemicals (e.g., oxidizing chemicals, antimicrobials, or other appropriate chemicals) into the water pond 1 without the need for direct intervention by personnel.

FIG. 6 provides an exemplary embodiment of a method of analyzing water health, represented in a yes-no flowchart. In the exemplary method, a combination of at least two measurements, e.g., ORP and DO, are utilized to trigger an alarm or alert indicating that conditions favorable to anaerobic microbial activity have been detected in the pond. In alternative embodiments and although not shown, it is to be appreciated that ORP and/or DO may be used in combination with H₂S measurements in a method similar to that represented by the yes-no flowchart of FIG. 6. In another alternative embodiment and not shown, one of ORP or DO measurements can be used to trigger the alarm. Using the combination of at least two independent measurements prevents too many unnecessary warning signals from being sent. This flowchart can be part of the mathematical model described above and installed in the remote data acquisition system, in a Cloud server, or in a remote server.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the present disclosure in any way. Rather, the foregoing detailed description will provide those skilled in the art with a convenient road map for implementing an exemplary embodiment of the present disclosure. It being understood that various changes may be made in the function and arrangement of elements described in an exemplary embodiment without departing from the scope of the present disclosure as set forth in the appended claims.

What is claimed is:

1. A system for analyzing pond water health, wherein the system comprises:
one or more of an oxidation-reduction potential sensor, a dissolved oxygen sensor, or an H₂S gas sensor;
a housing containing a control module, wherein the control module is in informational communication with the sensor(s);
wherein the system is positionable over a surface of a pond, proximal to the surface, or adapted to float on the surface.

2. The system of claim 1, comprising a combination of two or more different types of sensors.

3. The system of claim 1, wherein the system comprises at least one oxidation-reduction potential sensor and at least one dissolved oxygen sensor, and wherein the sensors are tethered to the housing of the system.

4. The system of claim 1, wherein the system comprises at least one H₂S gas sensor and wherein the at least one H₂S gas sensor is adapted to be positioned within 36 inches of the surface of the pond, when the system is disposed over or on the pond.

5. The system of claim 1, further comprising a water depth sensor.

6. The system of claim 5, comprising at least one oxidation-reduction potential sensor.

7. The system of claim 1, comprising a flotation feature to enable the system to float on the surface of the pond.

8. A method for analyzing pond water health of water pond, wherein the method comprises:
positioning the system of claim 1 over or on a surface of the pond;
obtaining data from the sensor(s) using the control module; and
generating an alert using data from the control module based on a determination that pond health is in decline.

9. The method of claim 8, wherein positioning the system over or on the surface comprises suspending the system over the surface of a pond, positioning the system proximal to the surface, or floating the system on the surface.

10. The method of claim 8, wherein obtaining the data comprises gathering data from the sensor(s) multiple times per day.

11. The method of claim 8, wherein the system comprises the H₂S gas sensor, and wherein the alert is generated based on detection of H₂S gas.

12. The method of claim 8, wherein obtaining the data comprises obtaining the data based on measurements made directly from the water in the pond in real time with the same sensors.

13. The method of claim 8, wherein obtaining the data comprises obtaining the data at different positions within the pond, and wherein the method further comprises comparing the data obtained from different parts of the pond using the control module.

14. The method of claim 8, wherein the system comprises the oxidation-reduction potential sensor, and wherein the alert is generated based on rate of change in oxidation-reduction potential measurements from the oxidation-reduction potential sensor.

15. The method of claim 8, wherein the system includes the oxidation-reduction potential sensor and/or the dissolved oxygen sensor, and wherein the system further comprises the H₂S gas sensor, and wherein the system obtains data from the oxidation-reduction potential sensor and/or the dissolved oxygen sensor below the water surface, and obtains data from the H₂S sensor taken over the surface.

16. The method of claim 8, wherein the control module autonomously generates and transmits an alert to a remote computer if any of the sensor measurements are outside of preset limits.

17. The method of claim 8, wherein the system includes the oxidation-reduction potential sensor and/or the dissolved oxygen sensor, and wherein the alert is generated based on a determination that conditions within the pond are conducive to anaerobic bacteria growth.

18. The method of claim 8, wherein the system includes the oxidation-reduction potential sensor and/or the dissolved oxygen sensor, wherein the system further comprises a water depth sensor, and wherein the method further comprises considering water depth measurements in combination with data from other sensors using the control module to determine water health and generate an alert.

19. The method of claim 18, wherein data from the water depth sensor indicates that water has been added to the pond, wherein data from the oxidation-reduction potential sensor and/or the dissolved oxygen sensor indicates a change over a period of time, and wherein the alert is generated based on data from the oxidation-reduction potential sensor and/or the dissolved oxygen sensor in combination with data from the water depth sensor.
